(19) United States
(12) Patent Application Publication
(10) Pub. No.: US 2004/0253289 A1
(43) Pub. Date: Dec. 16, 2004

(54) Natural plant compound with anti-HIV activity

(75) Inventors: Vladimir Khripach, Minsk (BY); Konstantin Altsivanovich, Minsk (BY); Vladimir Zabinskii, Minsk (BY); Mikhail Samusevich, Brooklyn, NY (US)

Correspondence Address: Mikhail Samusevich, Drebsk CompTech, Inc., 7201 19 Ave, 2 Floor, Brooklyn, NY, 11204

(73) Assignees: MIKONIK TECHNOLOGIES, LTD., Minsk District (BY); DREBSK COMPTECH, INC., Brooklyn, NY (US)

(21) Appl. No.: 10/711,162

(22) Filed: Aug. 28, 2004

(51) Int. Cl. A61K 31/415; A01N 43/52; A61K 47/00; A61K 35/78; A61K 9/20; A61K 9/48; A61K 9/14

(52) U.S. Cl. 424/422; 424/464; 424/465; 424/439; 424/451; 424/489; 424/725

(57) ABSTRACT

The present invention comprises a method for treatment of HIV-infection and related conditions, particularly AIDS, using plant hormone 24-epibrassinolide, anti-HIV efficacy of which is disclosed.

### FIELD OF THE INVENTION

The present invention relates to the treatment of viral infections. More particularly, the invention relates to the use of a compound known as steroidal plant hormone 24-epibrassinolide for the treatment of infections caused by virus responsible for human immune deficiency (HIV).

### BACKGROUND OF THE INVENTION

Within a short period of time the HIV/AIDS epidemic has emerged as one of the most serious health problems all over the world. The etiological agent of this complex disease resulting in a progressive destruction of the immune system is the human immunodeficiency virus. It belongs to the retrovirus family of viruses, which differ from the usual ones by the viral replication cycle. This cycle includes so-called reverse transcription, when RNA of retrovirus is used as a template for forming of the DNA. The obtained genetic material then puts viral replication instructions into effect.

Entering the body, HIV infects lymphocytes by attaching itself to receptors on their surface. The adhesion is an important stage of infection, which includes binding of glucoprotein 120 (gp120), HIV envelope glucoprotein, and CD4, a receptor existing on the surface of the host cell. Therefore, compounds inhibiting this process would be helpful for protection from HIV infection.

All known to date antiretroviral medicines belong to four main classes: nucleoside analogues, non-nucleoside reverse transcriptase inhibitors, protease inhibitors, and fusion inhibitors. Progress in the treatment of AIDS is rather slow because of extreme tendency of HIV to mutate to forms resistant to already developed antiviral drugs. That is why the best results were obtained from combination drug therapy which became standard in treatment of AIDS [Carpenter C. C. J., Fischl M. A., Hammer S. M., et al. Antiretroviral therapy for HIV infection in 1996. Recommendations of an international panel JAMA Vol. 276, P. 146-154, 1996] The main goal of such a therapy is to maintain level of HIV as low as possible. Decreasing the virus's amount results in reduction of immune system destruction. Taking into account that this approach implies long-term therapy, safety questions are very important. Besides, all the medications used nowadays in high active antiretroviral therapy (HAART) produce pronounced side effects, which are heavy endurable for patients very often. Another aspect of the problem is connected with the fact that AIDS is spread very widely in poor countries, and the proposed medicine should not be expensive. Thus, the first representative of the fusion inhibitors enfuvirtide (sold under the brand name Fuzeon) has been approved by the U.S. Food and Drug Administration in 2003 as anti-HIV medicine. It prevents binding of HIV to lymphocytes. When used with other anti-HIV medicines, Fuzeon reduces the amount of HIV in the blood and increases the number of CD4 cells. However, price of treatment is very high (about $20,000 per year).

With respect to safety problem, particularly interesting is the search for low toxicity natural compositions with anti-HIV activity. The fact that a certain compound is of natural origin (even if prepared synthetically, but structure of which fully corresponds to that for natural compound) provides a good prerequisite for elaboration of new medicines on its basis. It would allow using such anti-AIDS medicines over a long period with no or mild side effects or to use such compositions with anti-HIV properties in addition to the traditional anti-HIV preparations.

An example of naturally occurring low toxic compounds useful as antiviral agents are oxygenated derivatives of cholesterol (oxysterols) [Moog C., Aubertin A. M., Kirn A., Luu B. Oxysterols, but not cholesterol, inhibit human immunodeficiency virus replication in vitro. Antivir. Chem. Chemother., Vol. 9, No 6, P. 491-496, 1998] It was shown that 7β-hydroxycholesterol, 25-hydroxycholesterol, and 7β,25-dihydroxycholesterol inhibited viral replication at micromolar concentrations. The selectivity indexes for 7β-hydroxycholesterol and 25-hydroxycholesterol were quite modest (2 to 8) but reproducible; the 7β,25-dihydroxycholesterol showed antiviral properties at concentrations 13- to 25-fold lower than the highest concentration tested at which no toxicity was measurable.

Another possibility for search of the desirable pharmaceutical agents is presented by brassinosteroids, a new class of oxygenated sterols, which have been recently discovered in plants as hormones responsible for a wide spectrum of growth and adaptive reactions including phytoimmunity stimulation [Khripach V., Zhabinskii V., de Groot A. Twenty years of brassinosteroids: steroidal plant hormones warrant better crops for the XXI century. Annals of Botany, 2000, v.86, P. 447-447] Being constituents characteristic for all plant species known to date, brassinosteroids were inevitably consumed by animals with food throughout their co-evolution, and it could not be excluded that they have some regulatory functions in animals also. In addition to the data on low toxicity and absence of any direct and distant negative effects in animals, this circumstance indicate a possibility of their application to a solution of the task of the present invention without negative consequences for health of patients.

Along with antiviral and immunoactivating properties of brassinosteroids in plants there are data on their similar behavior in animals. Brassinosteroids were tested for antiviral activity against measles virus (MV) via a virus-yield reduction, herpes simplex and arenavirus and showed inhibitory activity [Ramirez J. A., Teme Centurion O. M., Gros E. G., Galagovsky L. R. Synthesis and bioactivity evaluation of brassinosteroid analogs. Steroids, Vol. 65, No 6, P. 329-337, 2000, Wachsman M. B., Lopez E. M., Ramirez J. A., Galagovsky L. R., Coto C. E. Antiviral effect of brassinosteroids against herpes virus and arena viruses. Antivir. Chem. Chemother. Vol. 11, No 1, P. 71-7, 2000, Wachsman M. B., Ramirez J. A., Galagovsky L. R., Coto C. E. Antiviral activity of brassinosteroids derivatives against measles virus in cell cultures. Antivir. Chem. Chemother. Vol. 13, No 1, P. 61-6, 2002]. 24-Epibrassinolide was shown to produce immunomodulating effect in Siberian sturgeon fry treated with its solution [Kolman H. The humoral effects of EPIN in Siberian Sturgeon (Acipenser Baeri Brandt). Arch. Pol. Fish., Vol. 9, Fasc. 1, P. 61-69, 2001]. Thus, γ-globulin level in the experimental group was from 10 to 65% higher than in the control, and this difference was statistically significant during certain period. Simultaneously, bacteriolytic activity of lysozyme increased up to 50% in treated group in comparison with the control fish. Although being known more than twenty years, brassinosteroids have not yet been investigated for treatment of HIV-infection [Khripach V. A., Zhabinskii V. N., Ae de Groot. Brassinosteroids—A New Class of Plant Hormones. Academic Press, 1999].

### SUMMARY OF THE INVENTION

The present invention relates to 24-epibrassinolide belonging to natural steroidal plant growth hormones. Extensive experiments in vitro have been carried out by the inventors, and they showed that 24-epibrassinolide is capable of reducing and in some cases even arresting the growth of the HIV in cultured infected cells. The compound showed a marked antiviral activity and inhibiting activity on viral replication in vitro.

Therefore, in a first aspect, the invention relates to the use of 24-epibrassinolide for the preparation of a medicine for the treatment of HIV-infection.

The in vitro antiviral activity of the 24-epibrassinolide was able to increase significantly the capability of the cell's living. More precisely, it was observed that the amount of the living cells in the infected culture treated with EBI was more than 50% higher in comparison with untreated control at 4-5^{th} days after infecting. Moreover, it has been observed a significantly decreased production of viral-specific antigens on the cell's surface at 3^{rd} day after infecting.

On the whole, the experimental data confirm that 24-epibrassinolide posseses an antiviral action against HIV. More particularly, 24-epibrassinolide reduces cytokilling properties of virus within the infected cells. Although the molecular mechanism of such effect has not yet fully been elucidated, there are no obstacles to recommend this compound for in vivo testing.

In addition to a marked antiviral effect of 24-epibrassinolide resulted in the increase of cell's resistance to the HIV influence, its inhibitory action on intracellular viral replication has been noted as well. Therefore, 24-epibrassinolide may be used in the treatment of HIV-infection and related conditions (“AIDS related complex”), both symptomatic and asymptomatic, or when exposure to HIV virus is suspected.

For the use in therapy, 24-epibrassinolide will be suitably formulated with pharmaceutically acceptable carriers and excipients. Suitable forms for the oral, parenteral or inhalatory administrations will be, for example, capsules, powders, granules, suppositories, solutions, suspensions, syrups, emulsions, injectable solutions, spray solutions or suspensions. The pharmaceutical compositions will be formulated according to conventional techniques, as described, for example, in Remington's Pharmaceutical Sciences Handbook, Mack Pub. Co., NY, USA, XVII Ed.

Dosages will vary depending on the severity of the disease, the general conditions and age of the patient, and will usually range in a daily dose from 0.03 to 200 micrograms per kilogram of body weight or in other doses which will be effective based on the results of clinic testing.

24-Epibrassinolide may be used in a combination with other anti-HIV preparations which will be currently available. Nucleoside and non-nucleoside reverse transcriptase inhibitors, protease inhibitors, and fusion inhibitors can be mentioned as examples.

Therefore, the invention also relates to pharmaceutical compositions in the form of combined preparations, for the simultaneous, separated or sequential use in the treatment of HIV-infection, comprising 24-epibrassinolide in addition to anti-HIV agent selected from the group of nucleoside and non-nucleoside reverse transcriptase inhibitors, protease inhibitors, fusion inhibitors, etc.

Taking into account low toxicity of 24-epibrassinolide as well as its other positive effects, like decreasing blood cholesterol, this compound may be used as food additive for HIV/AIDS prophylaxis. With respect to HIV, it means any measures which are aimed at preventing AIDS-related infections and illnesses.

### DETAILED DESCRIPTION OF THE INVENTION

According to the in vitro experiments using formazan assay, staining by trypan blue and indirect immunofluorescence, 24-epibrassinolide showed marked antiviral properties against human immunodeficiency virus.

24-Epibrassinolide is a plant hormone, which is an obligatory component of all plant tissues. Its content in plants is very low, ranging from 10⁻⁵ to 10⁻¹³% [Khripach V. A., Zhabinskii V. N., Ae de Groot. Brassinosteroids—A New Class of Plant Hormones. Academic Press, 1999]. The highest levels of 24-epibrassinolide were documented in reproductive tissues (seeds, pollen, etc.). This amount may be enough to provide pharmaceutical properties to certain plant stuff. Thus, pollen is used for many years as a drug in traditional medicine. Content of 24-epibrassinolide in this plant source is comparable in order with the doses used with the aims of the present invention. It may be speculated that being normal constituent of plants and a food component, 24-epibrassinolide is safe and non-toxic compound for a wide application in medicine and in prophylactic purposes. This assumption has got confirmations in different toxicological studies. They showed low acute toxicity of 24-epibrassinolide (LD₅₀ in mice is more than 1000 mg·kg⁻¹, in rats—more than 2000 mg·kg⁻¹) and confirmed no postponed negative effects in prolonged and chronic experiments.

Thus, 24-epibrassinolide can be considered as a non-toxic compound having anti-HIV properties that has been confirmed by experimental data.

### EXAMPLES

The following examples illustrate the invention in more detail. It is understood that those examples are not limitative for the scope of the invention.

Cell Line

In all experiments the suspensional T-lymphoblastoid cell line MT-4 was used. Passages have been carried out every three days for achieving the initial cell concentration at about 4-5·10⁵ cells ml⁻¹.

Virus

HIV-1_{zmb} high/rapid strain with the titer more than 6.0 log TCID₅₀ isolated in Belarus has been used in the tests.

Conditions

The tests have been carried out under therapeutic circuit when the testing compound and virus were put into the cell culture simultaneously.

### Example 1

Formazan Assay (FA)

Analysis is based on the metabolic reduction of the 3-(4,5-dimethylthiazol-2-yl)-2,5-diphenyltetrazolium bromide (MTT) that has been used to measure virus-induced cytopathic effects and cell proliferation [Mosmann T. Rapid coloremetric assay for cellular growth and survival application to proliferation cytotoxicity assays. J. Immunol. Meth., 1983, Vol. 65, P. 55-63]. The cellular reduction of MTT results in the formation of a colored, insoluble formazan products that must be solubilized by DMSO (dimethyl sulfoxide) prior to colorimetric determination. On the 5^{th} day after infecting, the resulting parameters including uninfected cell amount have been determined with the “Microwell system 230S” (Organon Teknika) at a test wavelength of 540 nm. The calculations were done in accordance with the formula of Pauwels R. C. et al. [Pauwels R. C., Balzarini J., Baba M. e.a. Rapid and automated tetrazolium-based colorimetric assay for detection of anti-HIV compounds. J. Virol. Meth., 1988, Vol. 20, P. 309-321].

It was found that 24-epibrassinolide had an ability to protect the living cells against HIV-1-cytopathic action in a concentration of 10^{-7.5}-10^{-9.5} M·l⁻¹ (10.0-0.1 ng·ml⁻¹).

### Example 2

Supravital Cell Staining by the Trypan Blue

This technique is based on the ability of living cells to impede the penetration of trypan blue dye into cell cytoplasm. The indicator was applied as a 0.2% concentration solution. On the 3^{rd} day after infecting, the amounts of living (unstained) and dead (blue stained) cells were counted under light microscope. Those probes were estimated as positive ones, where the amount of the living cells was 75% higher than in EBI-untreated virus control.

The calculations showed that 24-epibrassinolide was able to impede HIV-induced cell death at a concentration of 10⁻⁶-10^{-7.5} M·l⁻¹ (500.0-10.0 ng·ml⁻¹).

### Example 3

Indirect Immunofluorescence (IF)

The indirect immunofluorescence assay is a useful means for determining the number of cells productively infected with HIV [Johnson V. A., Byington R. E. HIV indirect immunofluorescence assay. In “Techniques in HIV Research”: Stockton Press, 1990. P. 87-91]. This assay is quantitative and can be done using living cells. The technique gives the possibility to determine HIV-specific antigens expressed on cell surface membranes of the virus-infected cells. On the 3^{rd} day after infecting, cells have been transferred onto wells of the laminated multiwell slides and fixed by cold (4° C.) acetone. Then the samples with fixed cells were treated by HIV-1-seropositive serum and then by FTC-conjugated sheep anti-human IgG. The resulting effect was estimated via evaluation of percentage of positive cells that was calculated using fluorescent microscope and taking into account 200 cells. Uninfected cells appear red, and infected cells exhibit yellow-green fluorescence. Those samples were estimated as positive ones, where the amount of fluorescent cells was 75% less than in EBI-untreated virus control.

The obtained results showed that 24-epibrassinolide decreased HIV-specific antigen production in the infected cells at a concentration of 10^{-6.5}-10⁻⁸ M·l⁻¹ (100.0-5.0 ng·ml⁻¹).

Each test done in three repetitions showed that average efficient concentration of EBI as anti-HIV agent in vitro is 10^{-7.5} M·l⁻¹ (10 ng ml⁻¹)

Pharmaceutical compositions that comprise one or more compounds of the invention may be formulated, as is well known in the prior art, such as by reference to known compilations as Remington's Pharmaceutical Sciences, Mack Publishing Co., Easton, Pa., USA. The dosage ranges for administration of the compounds of the invention are those needed to produce the desired effect without undue toxicity, whereby symptoms of infection are ameliorated.

24-Epibrassinolide can be applied in compositions at doses 0.03-200 micrograms per kilogram of body weight having in mind toxicological aspects obtained in the course of practical application. Other doses can not be excluded provided that their effectiveness will be proved by clinic trials.

The pharmaceutical composition may contain other pharmabiologically active compounds in a mixture with the compound of the invention, to treat (therapeutically or prophylactically) acquired immunodeficiency syndrome (AIDS). For example, other active compounds may include, but are not limited to, other antiviral compounds (e.g., AZT, ddC, TiBO derivatives, acyclovir, alpha-interferon), immunostimulants (e.g., various interleukins and cytokines), immunomodulators and antibiotics (e.g., antibacterial, antifungal, anti-pneumocystis agents), even when these do not show potent activity in the NCI Weislow protocol.

The preferred route of administration is oral, although other routes of administration are acceptable. The compounds may be mixed with inert materials for pharmaceutical efficacy as is known in the art. The compounds may be formulated in aqueous solution for intravenous (i.v.), intraperitoneal (i.p.), or subcutaneous (s.c.) administration. Topical applications include mixtures of the compounds with oils or fatty acid esters or as components of skin patches that are capable of delivering the drugs across the dermal layer. Aqueous solutions, or solutions in suitable carriers, may be administered intranasally.

Formulations include those suitable for oral, rectal, nasal, topical (including transdermal, buccal and sublingual), vaginal or parenteral (including subcutaneous, intramuscular, intravenous and intradermal) administration. The formulations may conveniently be presented in unit dosage form and may be prepared by any methods well known in the art of pharmacy. Such methods represent a further feature of the present invention and include the step of bringing into association the active ingredients with the carrier which constitutes one or more accessory ingredients. In general, the formulations are prepared by uniformly and intimately bringing into association the active ingredients with liquid carriers or finely divided solid carriers or both, and then if necessary shaping the product.

Formulations of the present invention suitable for oral administration may be presented as discrete units such as capsules, caplets, cachets or tablets each containing a predetermined amount of the active ingredients; as a powder or granules; as a solution or a suspension in an aqueous or non-aqueous liquid; or as an oil-in-water liquid emulsion or a water-in-oil liquid emulsion. The active ingredient may also be presented as a bolus, electuary or paste.

It should be understood that in addition to the ingredients particularly mentioned above the formulations of this invention may include other agents conventional in the art having regard to the type of formulation in question, for example, those suitable for oral administration may include such further agents as sweeteners, thickeners and flavoring agents.

What is claimed is:

1. A method of inhibition or treatment of Human Immunodeficiency Virus (HIV) infection using 24-epibrassinolide (EBI), a plant hormone of structural formula I belonging to brassinosteroid series.

2. The method of claim 1 wherein said 24-epibrassinolide is used as an active ingredient of pharmaceutical compositions for the prophylaxis or therapy of AIDS and related diseases.

3. A pharmaceutical composition for the aims of the present invention comprising a therapeutically effective amount of 24-epibrassinolide.

4. A pharmaceutical composition containing an effective amount of 24-epibrassinolide in a combination with other anti-HIV agents.

5. The pharmaceutical composition according to claim 4, wherein the anti-HIV agents are selected from at least one group of inhibitors, such as protease inhibitors, reverse transcriptase inhibitors, or integrase inhibitors.

6. A food supplement for the aims of the present invention containing an effective amount of 24-epibrassinolide.

7. The food supplement of claim 6 wherein said pharmaceutical or over-the-counter preparation is a tablet, capsule, powder, suspension, solution, or aqueous emulsion.

8. The food supplement of claim 6 incorporated into a food material.

9. A vaginal tract protecting HIV-inhibiting composition containing 24-epibrassinolide as an active ingredient.

10. The composition of claim 9 wherein said 24-epibrassinolide is a component of cream, vaginal insertion, or coating on a condom.
